(19) United States
(12) Patent Application Publication
(10) Pub. No.: US 2026/0340823 A1
(43) Pub. Date: Oct. 29, 2026

(54) SYSTEM AND METHOD FOR PREDICTION OF STENT EXPANSION FROM ANGIOGRAPHY VIDEO AND TABULAR CLINICAL DATA

(71) Applicant: MANAN SAMIR PANCHOLY, CLARKS SUMMIT, PA (US)

(72) Inventor: MANAN SAMIR PANCHOLY, CLARKS SUMMIT, PA (US)

(21) Appl. No.: 19/697,575

(22) Filed: Jun. 4, 2026

(51) Int. Cl. G06T 7/00; A61B 34/10

(52) U.S. Cl. CPC G06T 7/0012; A61B 34/10; A61B 2034/108; G06T 2207/10016; G06T 2207/10101; G06T 2207/10116; G06T 2207/10132; G06T 2207/20081; G06T 2207/30052

(57) ABSTRACT

The disclosure relates to methods and systems for predicting coronary stent expansion using cineangiography and structured clinical variables during PCI. A machine learning model processes a short post-stent angiography clip together with clinical variables to output a predicted stent expansion metric as a continuous value, an expansion adequacy category, or both. The model may be trained using paired angiography and intravascular imaging data, including intravascular-imaging-derived expansion labels, and may be trained with multiple auxiliary tasks such as vessel identity, angiographic projection, and intravascular-imaging-derived lesion metrics. The present disclosure addresses the challenge of objectively assessing stent expansion in PCI laboratories where intravascular imaging is unavailable or impractical while supporting real-time intervention guidance and retrospective quality review.

### FIELD

In part, the disclosure relates to methods, devices, systems, and non-transitory computer-readable media for predicting vascular stent expansion using extraluminal or intraluminal x-ray angiography data and patient clinical variables, including by training and deploying one or more machine learning models.

In one embodiment, the disclosure relates to predicting a post-deployment stent expansion metric (also referred to herein as “stent expansion,” “percent expansion,” “expansion percentage,” or “expansion adequacy”) from a post-stent cineangiography video segment and one or more structured clinical variables, without requiring intravascular imaging to be performed at the time of prediction. In one embodiment, the intravascular imaging modality is intravascular optical coherence tomography (OCT), intravascular ultrasound (IVUS), or another intravascular imaging modality.

### BACKGROUND

In percutaneous coronary intervention (PCI), a stent is deployed to treat an atherosclerotic coronary lesion. Following deployment, inadequate stent expansion is associated with adverse outcomes and may require additional optimization steps such as post-dilation, lesion modification, placement of an additional stent, or other adjunctive therapy.

Intravascular imaging modalities, including intravascular optical coherence tomography (OCT) and intravascular ultrasound (IVUS), are widely used to assess stent expansion and identify under-expansion, malapposition, edge dissection, and lesion characteristics (including calcification) that affect expansion. However, intravascular imaging is not available in many catheterization laboratories and may be impractical in certain settings due to cost, time, logistics, and expertise requirements.

Angiography is a widely available imaging modality in PCI laboratories, but angiography is limited as a low-resolution, two-dimensional projection of a three-dimensional vessel. Important predictors of under-expansion, such as calcification distribution and depth, can be difficult to appreciate using angiography alone.

Accordingly, a need exists for systems and methods that can provide an objective estimate of post-stent expansion quality in angiography-only workflows, including in real time or substantially real time during PCI.

A further need exists for systems that can be trained using higher-resolution reference labels, such as intravascular-imaging-derived expansion measurements, yet deployed using only universally available data sources, such as angiography and basic clinical variables.

A further need exists for machine learning systems that can generalize across operators, projections, equipment vendors, and lesion morphologies, including by using training strategies that improve robustness when labeled datasets are relatively limited.

The present disclosure addresses these challenges and others.

### SUMMARY

A system of one or more computers can be configured to perform particular operations or actions by virtue of having software, firmware, hardware, or a combination thereof installed on the system that in operation causes the system to perform the actions.

One or more computer programs can be configured to perform particular operations or actions by virtue of including instructions that, when executed by a data processing apparatus, cause the apparatus to perform the actions.

One general aspect includes a method of predicting a stent expansion metric using angiography video and clinical variables. The method includes receiving, by one or more processors, a post-stent cineangiography video segment of a vessel; receiving, by the one or more processors, one or more structured clinical variables; applying, by the one or more processors, a trained machine learning model to the cineangiography video segment and the clinical variables; and outputting, by the one or more processors, a predicted stent expansion metric.

In one embodiment, the predicted stent expansion metric is a continuous value representing percent stent expansion relative to a reference, target, or guideline-based metric, wherein the continuous value is output as a scalar or as a value normalized to a predetermined range.

In one embodiment, the predicted stent expansion metric is an ordinal classification output that categorizes expansion adequacy into two or more ordered categories, including without limitation categories such as subpar, adequate, or optimal.

In one embodiment, the trained machine learning model is trained using paired angiography and intravascular imaging data, wherein ground-truth stent expansion labels are derived from intravascular imaging pullback or acquisition analysis, and wherein the trained model is deployed without requiring intravascular imaging at inference time. In one embodiment, the intravascular imaging modality used for training labels is intravascular optical coherence tomography, intravascular ultrasound, or another intravascular imaging modality.

In one embodiment, the trained machine learning model is a multimodal model comprising (i) a video encoder configured to transform angiography video frames into an embedding and (ii) a clinical feature encoder configured to transform clinical variables into an embedding, and (iii) a fusion module configured to combine the embeddings into a fused representation used to generate the predicted stent expansion metric.

In one embodiment, the video encoder comprises a spatiotemporal neural network including a three-dimensional convolutional neural network (3D CNN), a 2+1D CNN, a recurrent neural network configured to model temporal relationships, a video vision transformer, or combinations thereof.

In one embodiment, the clinical feature encoder comprises a multilayer perceptron (MLP), a generalized linear model, a gradient-boosted tree model, an embedding lookup model, or combinations thereof.

In one embodiment, the model is trained using a multi-task learning objective that includes a primary loss associated with predicting the stent expansion metric and one or more auxiliary losses associated with one or more auxiliary prediction tasks.

In one embodiment, the auxiliary prediction tasks include predicting a treated vessel identity (for example, LAD, LCx, RCA), predicting an angiographic projection category, and predicting one or more intravascular imaging-derived lesion metrics (including without limitation calcium metrics).

In one embodiment, multi-task training improves robustness of the learned video representation by regularizing the video encoder to learn features predictive of (i) vessel anatomy, (ii) projection geometry, and (iii) lesion morphology, in addition to the primary stent expansion target.

In one embodiment, the angiographic projection category is labeled using DICOM metadata associated with a C-arm or image acquisition system or by clinician labeling, and is used as an auxiliary supervision signal during training.

In one embodiment, the system outputs the predicted stent expansion metric to a user interface in a catheterization laboratory, wherein the user interface provides decision support for stent optimization (including without limitation whether to consider further post-dilation, placement of an additional stent, or selection of a post-dilation balloon diameter or inflation pressure).

In one embodiment, the system operates in real time or substantially real time by processing a short cineangiography segment, for instance a segment comprising approximately 4 to 10 seconds of video, wherein the number of sampled frames is determined as a function of the acquisition frame rate so as to maintain consistent temporal coverage across cases acquired at different frame rates (often approximately 7.5 to 30 frames per second).

In one embodiment, the system is used in retrospective quality assurance, operator training, or research workflows by processing stored angiographic videos and outputting predicted stent expansion values for review.

In one embodiment, the system further stores the predicted stent expansion metric, the input angiography segment, and associated metadata in electronic storage for later retrieval and auditing.

In one embodiment, the model is trained using supervised learning on a dataset of paired angiography and intravascular imaging cases. In one embodiment, the dataset comprises on the order of thousands of cases.

In one embodiment, the training procedure includes selecting frames centered on a time period following stent deployment, normalizing image intensities, resizing frames to a consistent resolution, and sampling a fixed number of frames to form a video clip input.

In one embodiment, the system further includes one or more preprocessing components configured to identify or confirm that an angiography segment corresponds to a post-stent state, including by detecting radiopaque stent appearance, device markers, balloon markers, contrast dynamics, or combinations thereof.

In one embodiment, the output includes a confidence score, uncertainty estimate, or probability distribution over expansion categories, wherein uncertainty can be used to determine when to recommend confirmatory imaging or additional assessment.

In one embodiment, the system is configured to operate on single-view angiography or multi-view angiography. In one embodiment, a dual-stream or multi-stream encoder is used for multi-view inputs, and the streams are fused by concatenation, attention, or other fusion.

In one embodiment, the invention applies to coronary arteries. In other embodiments, the invention applies to other vessels and stents, including peripheral arterial stents, carotid stents, renal stents, or other endovascular implants.

Although the disclosure relates to different aspects and embodiments, it is understood that the different aspects and embodiments disclosed herein can be integrated, combined, or used together as a combination system, or in part, as separate components, devices, and systems, as appropriate.

The foregoing summary is illustrative only and is not intended to limit the scope of the claims.

### BRIEF DESCRIPTION OF DRAWINGS

The patent or application file contains at least one drawing executed in color. Copies of this patent or patent application publication with color drawing(s) will be provided by the Office upon request and payment of the necessary fee.

The figures are not necessarily to scale; emphasis instead is generally placed upon illustrative principles. The figures are to be considered illustrative in all aspects and are not intended to limit the disclosure, the scope of which is defined only by the claims.

FIGS. 1a, 1b, and 1c form a schematic diagram of an example multimodal, multi-task machine learning model architecture for predicting stent expansion from angiography video and clinical variables according to an illustrative embodiment of the disclosure. FIG. 1a outlines the steps from initial data input to model predictions. FIG. 1b outlines the multi-task training objective given model predictions. FIG. 1c outlines the model's gradient flow and parameter updates during backpropagation.

FIG. 2 is a workflow diagram illustrating an example of the dataset creation process, including the extraction of raw video and tabular data from the angiography system, intravascular imaging viewer, and electronic health record, human assessment and measurement of necessary endpoints from video data, and multimodal data harmonization procedures according to an illustrative embodiment of the disclosure.

FIG. 3 is a workflow diagram illustrating an example of the training procedure for the ANGIO-EXPAND model, including cross-validation of performance across the processed dataset, model selection, and model export for later clinical deployment according to an illustrative embodiment of the disclosure.

FIGS. 4a and 4b form a workflow diagram illustrating an example of an end-to-end process for clinical deployment for predicting stent expansion and iterative model improvement according to an illustrative embodiment of the disclosure. FIG. 4a outlines the primary use case of the model in clinical practice: real-time stent optimization guidance. FIG. 4b outlines the secondary use cases of the model in clinical practice: trainee instruction and model retraining.

### DETAILED DESCRIPTION

The following paragraphs describe certain explanatory embodiments. Other embodiments may include alternatives, equivalents, and modifications. Additionally, the explanatory embodiments may include several novel features, and a particular feature may not be essential to some embodiments of the devices, systems, and methods that are described herein.

In part, the disclosure relates to a system (also referred to herein as “ANGIO-EXPAND”) configured to predict a stent expansion metric using angiography video acquired after a stent is deployed, together with structured clinical variables available during a PCI procedure.

In one embodiment, “angiography” refers to x-ray imaging (including fluoroscopy) performed with intravascular contrast injection to visualize blood vessels. “Cineangiography” refers to a time series of angiography frames, for instance acquired at approximately 7.5 to 30 frames per second.

In one embodiment, “intravascular imaging” refers to any imaging modality that acquires images from within a blood vessel using an intravascular catheter, including without limitation intravascular optical coherence tomography (OCT), intravascular ultrasound (IVUS), near-infrared spectroscopy combined with intravascular ultrasound (NIRS-IVUS), and optical frequency domain imaging (OFDI). In one embodiment, intravascular imaging includes pullback or stationary acquisitions producing cross-sectional frames and longitudinal reconstructions. In one embodiment, “OCT” refers to intravascular optical coherence tomography, including frequency-domain OCT, swept-source OCT, and optical frequency domain imaging. In one embodiment, “IVUS” refers to intravascular ultrasound, including grayscale IVUS, high-definition IVUS, and IVUS combined with other modalities such as near-infrared spectroscopy.

In one embodiment, “stent expansion” refers to a quantitative metric that characterizes the degree of expansion of a deployed stent relative to a target, reference vessel segment, manufacturer nominal size, or guideline-based expansion metric. In one embodiment, the stent expansion metric is derived from intravascular imaging by measuring one or more cross-sectional areas, diameters, or other dimensional measures along the stented segment and comparing to a reference.

In one embodiment, a “stent expansion percentage” is computed as a ratio or percentage, for example a minimum stent area (MSA) divided by a reference lumen area, an average reference lumen area, or a target stent area, optionally multiplied by 100 to express a percentage. Other intravascular imaging-derived stent expansion indices may be used, including indices derived from intravascular OCT, IVUS, or other intravascular imaging modalities.

In one embodiment, the system is configured to operate without intravascular imaging at inference time. Intravascular imaging is used to generate training labels and auxiliary labels, and angiography is used as the primary inference input. In one embodiment, when multiple modalities are combined within a single training dataset, expansion labels are harmonized across modalities through calibration of cross-sectional area measurements, since IVUS and intravascular OCT are known to produce systematically different lumen and stent area measurements on the same vessel.

In one embodiment, the system is deployed in a PCI laboratory and receives angiography data from an angiography system, such as a catheterization laboratory C-arm system. In one embodiment, the system receives angiography data via DICOM transfer, video frame capture, or another interface.

In one embodiment, the system receives structured clinical variables from an electronic health record (EHR), hemodynamic system, procedure log, structured case report form, or manual entry by an operator.

In one embodiment, structured clinical variables include one or more of: age, sex, height, weight, diabetes status, chronic kidney disease status, dialysis history, dyslipidemia, smoking history, prior myocardial infarction, prior PCI, prior coronary artery bypass grafting, lesion location, and other comorbidities or procedural variables. In one embodiment, structured inputs further include acquisition and calibration metadata obtained from DICOM headers or operator entry, including without limitation pixel spacing, magnification factor, source-to-detector distance, source-to-patient distance, frame rate, and C-arm angulation, wherein such metadata is provided to the clinical feature encoder, to a separate metadata encoder, or directly to the fusion module.

In one embodiment, the system includes one or more processors, memory, and one or more software modules configured to perform preprocessing, model inference, and output display. In one embodiment, the system further includes one or more AI processors such as a graphical processing unit (GPU), tensor processing unit (TPU), or other accelerator.

In one embodiment, the system operates locally within the PCI laboratory. In one embodiment, the system operates remotely, such as through a client-server architecture, edge computing architecture, or cloud-based architecture, provided that latency and data security requirements are satisfied.

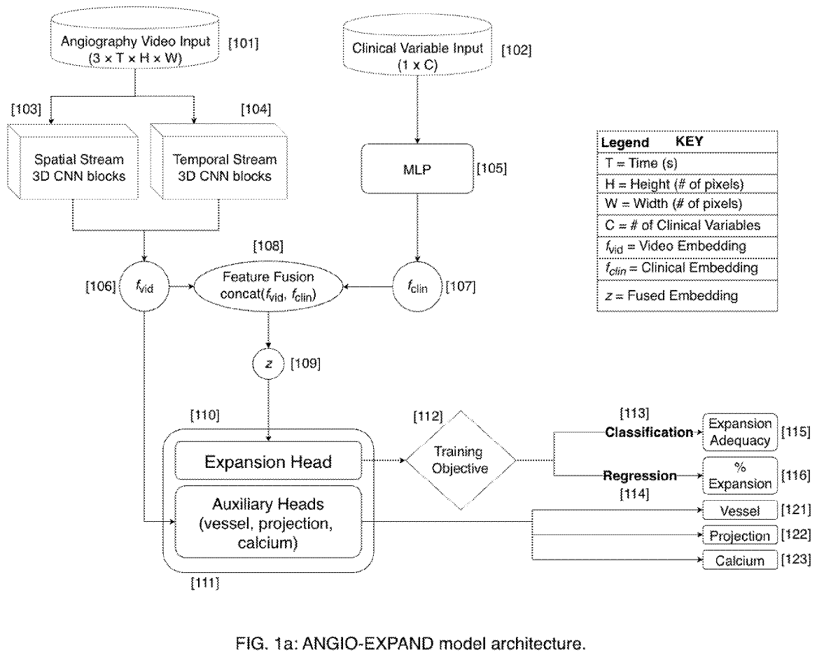

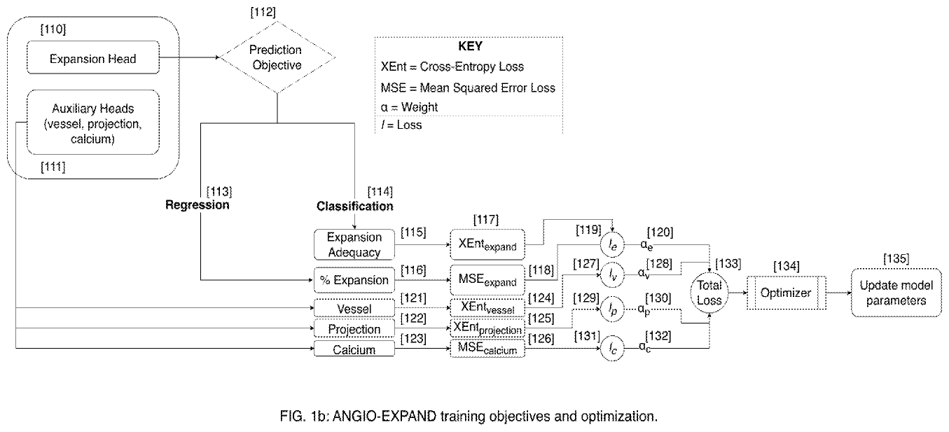

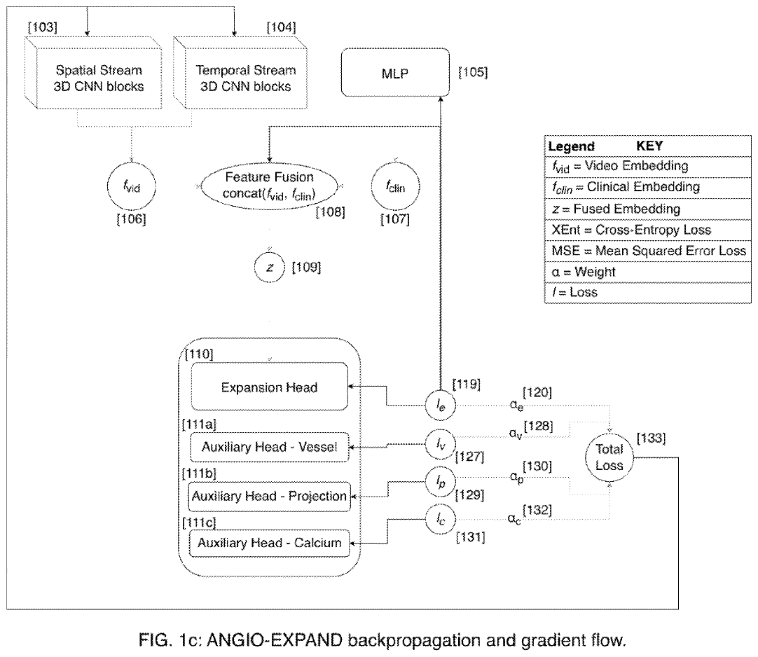

FIG. 1a illustrates an example model architecture. In one embodiment, an angiography video input 101 is provided as a tensor comprising a sequence of frames with the dimensions. Frames are represented in grayscale or multi-channel form.

In one embodiment, the model includes a video encoder configured to extract spatial (block 103) and temporal (block 104) features from the angiography clip and produce a video embedding vector 106. In one embodiment, the video encoder includes a 3D CNN backbone.

In one embodiment, the 3D CNN backbone includes one or more of: X3D-style blocks, I3D-style blocks, R(2+1)D-style blocks, residual blocks, separable convolutions, or other architectures configured for video representation learning.

In one embodiment, the video encoder includes a video transformer backbone, such as Swin3D or VideoMAE. The transformer processes frame patches, tubelets, or tokens to generate a video embedding.

In one embodiment, the video encoder generates an embedding vector 106 representing the angiographic appearance of a stented segment, including features related to contrast flow, vessel motion, stent radiopacity patterns, lesion morphology, and projection geometry.

In one embodiment, the model is initialized from pretrained weights. In one embodiment, the video encoder is pretrained using self-supervised learning on unlabeled angiography videos and then fine-tuned on paired angiography-intravascular-imaging labels.

In one embodiment, the model further includes a clinical feature encoder configured to process structured clinical variables 102 into an embedding. In one embodiment, the clinical feature encoder comprises an MLP system 105 that outputs a fixed-length clinical embedding vector 107.

In one embodiment, clinical variables are normalized prior to encoding, including by z-scoring continuous variables and one-hot encoding categorical variables. In one embodiment, missing values are handled by imputation or by including missingness indicators.

In one embodiment, a fusion module 108 combines the video embedding 106 and the clinical embedding 107 into a fused embedding vector 109. In one embodiment, fusion is performed by concatenation followed by one or more fully connected layers.

In one embodiment, when biplane or multi-view acquisitions are available, C-arm angulation metadata for each view is provided as an additional input to the fusion module 108, and each view's embedding vectors are fused using a geometry-aware mechanism that accounts for their relative projection angles.

In one embodiment, fusion is performed using attention-based fusion, gating, multiplicative interactions, or other multimodal fusion mechanisms.

In one embodiment, prediction heads process embedding vectors as input to output the model's ultimate predictions. In one embodiment, prediction heads are linear projections. In another embodiment, prediction heads are shallow MLPs.

FIG. 1b illustrates an example learning paradigm, including training objectives and optimization, for the model. In one embodiment, a primary (expansion) prediction head 110 receives the fused embedding and outputs a predicted stent expansion metric according to the prediction objective 112. In one embodiment, the primary prediction head produces a scalar output 116 for regression 114.

In one embodiment, the primary prediction head outputs logits for classification 113 into ordered categories of expansion adequacy 115. In one embodiment, an ordinal cross-entropy loss 117 is used.

In one embodiment, the model is trained using multi-task learning, wherein auxiliary prediction heads 111 branch from the video embedding. In another embodiment, the auxiliary prediction heads branch from the fused embedding.

In one embodiment, an auxiliary head predicts vessel identity 121. In one embodiment, vessel identity classes include LAD, LCx, and RCA. Other vessel taxonomies may be used.

In one embodiment, an auxiliary head predicts angiographic projection 122. In one embodiment, projection classes are determined by binning C-arm angulation metadata into a finite set of categories.

In one embodiment, an auxiliary head predicts one or more intravascular imaging-derived lesion metrics 123. In one embodiment, intravascular imaging-derived lesion metrics include one or more continuous calcium-related metrics such as maximum calcium arc, calcium length, calcium thickness, calcium depth, a composite calcium score, or other quantitative parameters. In one embodiment, the intravascular imaging modality from which the lesion metrics are derived is intravascular OCT, IVUS, or another intravascular imaging modality, wherein the specific lesion metrics available depend on the modality. For example, calcium thickness is typically measurable on OCT but not on IVUS, while attenuated plaque burden is typically measurable on IVUS.

In one embodiment, auxiliary tasks are used during training to encourage the video encoder to learn representations that capture clinically meaningful factors correlated with stent expansion.

In one embodiment, the primary expansion loss includes a regression loss 118 such as mean squared error (MSE), mean absolute error (MAE), Huber loss, or another regression loss. In one embodiment, the primary expansion loss includes an ordinal classification loss 117 such as ordinal cross-entropy loss.

In one embodiment, auxiliary losses include cross-entropy losses 124 and 125 for categorical prediction tasks (vessel identity and projection) and a regression loss 126 for continuous prediction tasks (lesion metrics).

In one embodiment, a total loss 133 is calculated as a weighted sum of the primary expansion and auxiliary losses. In one embodiment, the loss weights 120, 128, 130, and 132 are selected empirically, selected using validation performance, selected by a hyperparameter search, or selected using an adaptive loss-weighting method. In one embodiment, the adaptive loss-weighting method comprises uncertainty-based task weighting (namely, learning per-task homoscedastic uncertainties), GradNorm, PCGrad, or another gradient-balancing method, such that the primary expansion task is not dominated by auxiliary tasks of differing scale or noise level.

In one embodiment, the model's training objective is a weighted sum of a primary expansion loss 119 and one or more auxiliary losses 127, 129, and 131. The parameters of the model are updated in process 135 using an optimizer 134. In one embodiment, the adaptive momentum optimizer with decoupled weight decay (AdamW) is used. In another embodiment, the MomentUm Orthogonalized by Newton-Schulz (Muon) optimizer is used.

FIG. 1c illustrates an example backpropagation and gradient flow paradigm for the model. In one embodiment, translucent arrows in FIG. 1c reproduce the forward-pass connections previously depicted in FIG. 1a and FIG. 1b, and are included in FIG. 1c for visual reference only without representing any additional computational step. In one embodiment, opaque arrows in FIG. 1c represent gradient propagation during backpropagation, wherein each opaque arrow drawn from a source node to a target node implies application of the chain rule sequentially through every module with learnable parameters that lies between the source and the target along the original forward path, in the reverse direction of the forward pass. In one embodiment, the source of each opaque arrow is a loss node or the total loss node, and the target of each opaque arrow is a module group whose learnable parameters are updated using the gradient signal carried by that arrow.

In one embodiment, an opaque arrow originates at the primary expansion loss node 119 and terminates at the clinical feature encoder 105, wherein the gradient propagates in reverse through the expansion head 110, the fused embedding 109, the fusion module 108, and the clinical embedding 107 before reaching the clinical feature encoder 105. In one embodiment, this opaque arrow represents the singular gradient signal received by each of the clinical feature encoder 105, the clinical embedding 107, the fusion module 108, the fused embedding 109, and the expansion head 110, since each of these modules is downstream of the primary expansion loss alone in the forward computation graph.

In one embodiment, an opaque arrow originates at the vessel auxiliary loss node 127 and terminates at the vessel auxiliary head 111a, an opaque arrow originates at the projection auxiliary loss node 129 and terminates at the projection auxiliary head 111b, and an opaque arrow originates at the calcium auxiliary loss node 131 and terminates at the calcium auxiliary head 111c. In one embodiment, each such opaque arrow represents the singular gradient signal received by its corresponding auxiliary head, since each auxiliary head is downstream of exactly one auxiliary loss in the forward computation graph and therefore receives no gradient contribution from any other loss term.

In one embodiment, an opaque arrow originates at the total loss node 133 and terminates at the video encoder, including the spatial stream 103 and the temporal stream 104, wherein the gradient propagates in reverse through the video embedding 106 before reaching the spatial and temporal streams. In one embodiment, the gradient signal carried by this opaque arrow is the singular combined gradient signal received by the video encoder, since the video encoder lies downstream of all four loss terms in the forward computation graph by virtue of contributing to the fused embedding 109 (through the fusion module 108) and to each of the auxiliary heads 111a, 111b, 111c (directly from the video embedding 106). In one embodiment, the combined gradient signal received by the video encoder reflects contributions from the primary expansion loss, the vessel auxiliary loss, the projection auxiliary loss, and the calcium auxiliary loss, combined according to the weighting scheme used to construct the total loss 133.

In one embodiment where the weighting scheme uses static weights, validation-selected weights, hyperparameter-searched weights, or uncertainty-based task weighting, the total loss weighting scheme operates on the construction of the total loss 133 in the forward direction, such that the gradient signal received by the video encoder along the opaque arrow originating at the total loss node 133 inherits the per-task weighting through the chain rule without requiring any post-hoc modification of gradients during the backward pass.

In one embodiment, the gradient signals received by each module group along the opaque arrows depicted in FIG. 1c are consumed, as depicted in FIG. 1b, by the optimizer 134 to update the learnable parameters of the corresponding module group in process 135. In one embodiment, the opaque arrows depicted in FIG. 1c collectively cover every module group with learnable parameters in the model, such that the video encoder, the clinical feature encoder, the fusion module, the expansion head, and each auxiliary head receive exactly one singular gradient signal per training step. In one embodiment, the asymmetry between the singular combined gradient signal received by the video encoder and the per-loss gradient signals received by the remaining module groups reflects the position of each module group in the forward computation graph, wherein modules downstream of multiple loss terms receive a combined gradient signal and modules downstream of a single loss term receive a per-loss gradient signal.

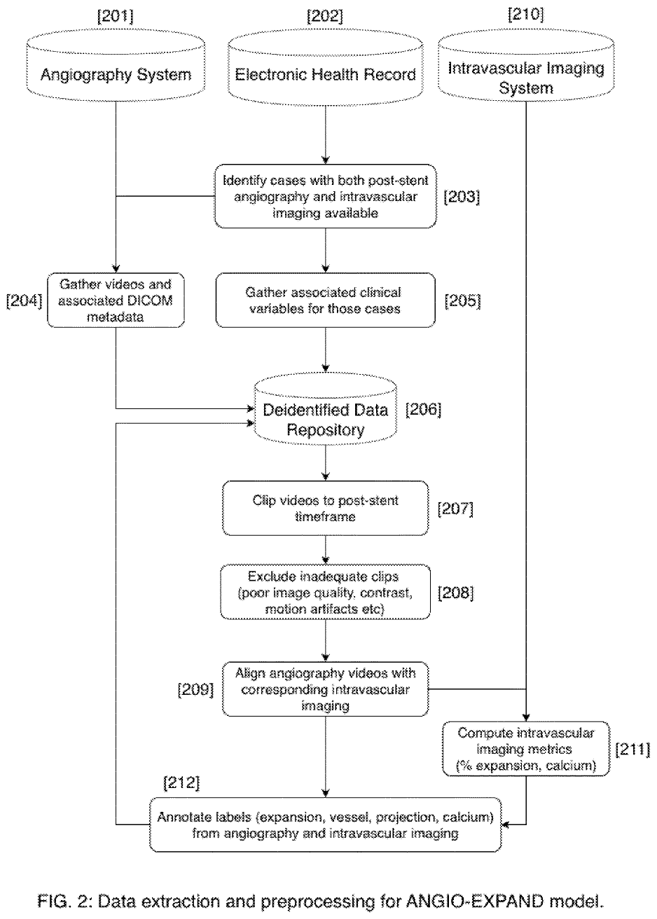

FIG. 2 illustrates an example data extraction and preprocessing procedure. In one embodiment, cases with both angiography and intravascular imaging acquisitions available post-stent-deployment are identified within EHR system 202. The associated clinical variables from these cases are retrieved from system 202. Values are stored in a secure, deidentified data repository system 206 as a table, with rows representing individual cases and columns representing variables.

In one embodiment, angiography videos and their associated DICOM metadata 204 are retrieved from the angiography system 201. Frames are represented in grayscale or multi-channel formats. Videos corresponding to a given case are stored in individual folders in the data repository 206. The paths to these folders for each case are stored in a column of the table alongside clinical variables.

In one embodiment, the angiography video is preprocessed per process 207 by selecting a contiguous or sampled subset of frames corresponding to a time window after stent deployment. In one embodiment, the frames are resized to a standard resolution and intensity-normalized. Those videos with poor image quality, inadequate contrast injection, or severe motion artifacts are excluded from the data repository 206.

In one embodiment, the video is additionally preprocessed by cropping around a region of interest corresponding to the treated vessel segment. In one embodiment, the region of interest is selected manually. In one embodiment, the region of interest is selected automatically using a vessel segmentation model, a tracking model, a centerline extraction model, or combinations thereof.

In one embodiment, the intravascular imaging system 210 is used per process 211 to compute metrics that correspond to each case in the data repository 206. In one embodiment, the intravascular imaging system 210 is an intravascular OCT pullback system, an IVUS system, or another intravascular imaging system.

In one embodiment, the cases are annotated via process 212 such that the values of stent expansion, calcium burden, angiography video projection, and vessel identity for each case are stored in additional columns in the data table.

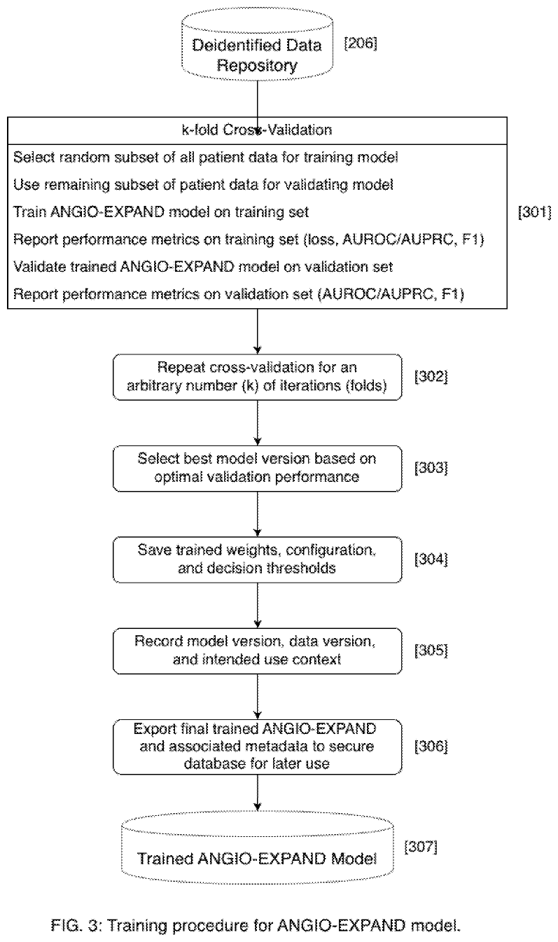

FIG. 3 illustrates an example procedure for model training and export. In one embodiment, the training procedure includes splitting a dataset into training, validation, and test subsets. In one embodiment, k-fold cross-validation 302 is used per process 301. k-fold cross-validation is a machine learning technique used to evaluate model performance by dividing the dataset into equal-sized subsets, iteratively training the model on k-1 subsets, and testing it on the remaining subset until all k subsets have been used as the test set.

In one embodiment, the training procedure includes data augmentation applied to angiography clips, including without limitation brightness perturbation, additive noise, cropping, scaling, and temporal sampling.

In one embodiment, augmentations are constrained to preserve clinically meaningful features, for example avoiding transformations that destroy temporal contrast-flow patterns.

In one embodiment, cross-validation is repeated for a specified number of iterations per process 302. The model with the best average performance on the validation set across folds is selected per process 303.

In one embodiment, the model weights, hyperparameters, calibration, model version, data version, and intended use case are stored, per processes 304-306, in database 307's non-transitory memory and later deployed to a PCI laboratory system for inference.

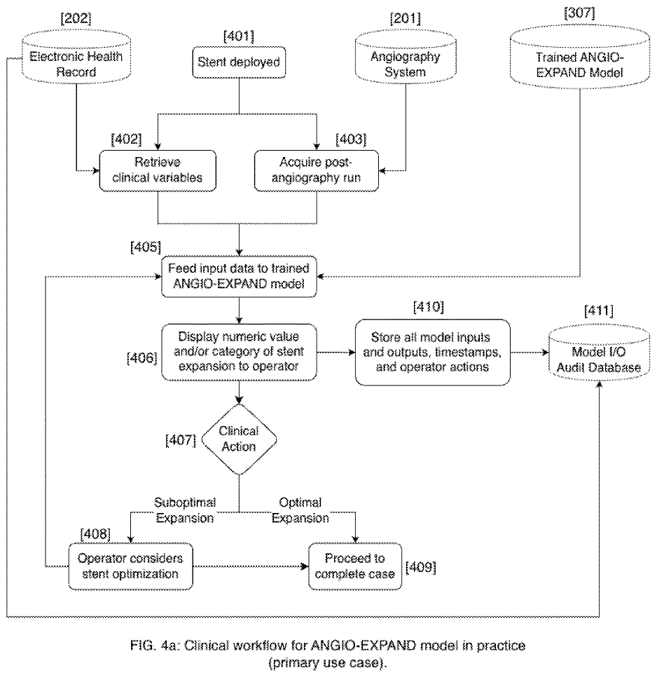

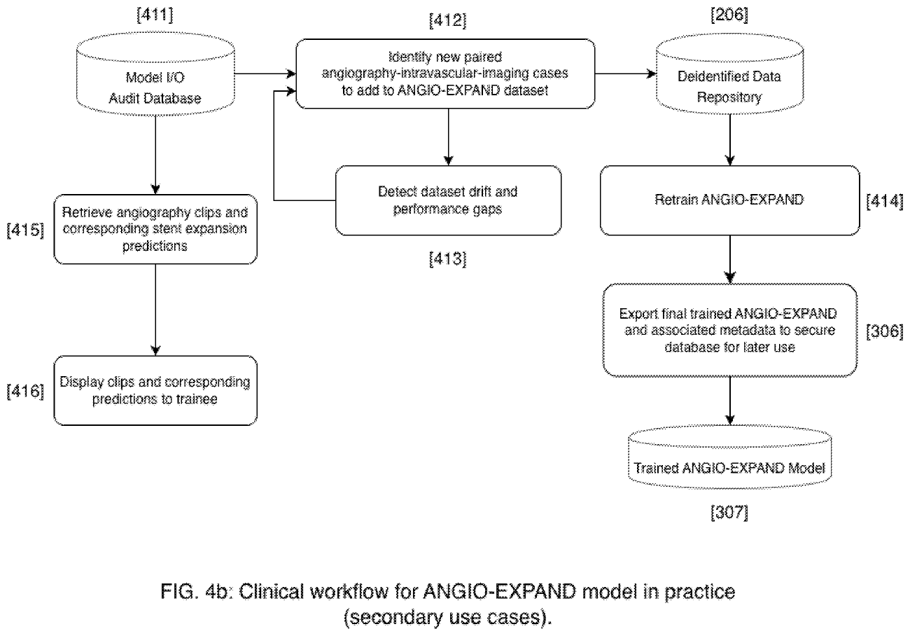

FIG. 4a illustrates an example deployment of ANGIO-EXPAND in a PCI laboratory system for the purpose of stent optimization guidance. In one embodiment, after stent deployment 401, the patient's clinical variables 403 and post-stent angiography video clip 405 are retrieved from the EHR system 202 and angiography system 201, respectively.

In one embodiment, the most recently trained model is remotely retrieved from database 307. In one embodiment, the most recently trained model exists locally on a non-transitory computer-readable medium in the PCI laboratory. The model processes the patient's clinical variables and post-stent angiography clip as input per process 405.

In one embodiment, during inference the system processes a single post-stent angiography clip acquired in a standard view. In one embodiment, the system processes a multi-view set of clips.

In one embodiment, the system outputs the predicted stent expansion metric 406 to a display, including in an overlay, widget, numeric readout, or structured report.

In one embodiment, the system outputs an alert when predicted expansion is below a threshold. The threshold is user-defined, guideline-based, or institution-defined.

In one embodiment, the system outputs to the operator a recommendation to consider further stent optimization via process 408, including without limitation additional post-dilation, additional lesion preparation, placement of one or more additional stents, or confirmatory intravascular imaging if available. In one embodiment, the recommendation is presented within a time window that permits the operator to select, before the patient leaves the catheterization laboratory, one or more of a post-dilation balloon diameter, a post-dilation balloon inflation pressure, or placement of an additional stent. After each attempt at stent optimization, the new post-dilation angiography clip is fed as input to the model once again, repeating processes 405-408 as part of an iterative optimization process. Once stent expansion is adequate, as determined by the operator, the operator proceeds to complete the case per process 409.

In one embodiment, the system outputs an explanation interface that displays contributory signals such as predicted calcium severity, vessel identity, or projection metadata, to assist clinician interpretation and, ultimately, clinical action 407.

In one embodiment, per process 410, the system stores its inputs (angiography clip metadata and clinical variables) and outputs (predicted stent expansion) in a secure database 411.

FIG. 4b illustrates an example deployment of ANGIO-EXPAND in a PCI laboratory system for the purposes of clinical trainee instruction and iterative model improvement. In one embodiment, new stent procedures with paired angiography and intravascular imaging data available are periodically added to the deidentified data repository 206 per process 412. Per process 413, inclusion criteria for these additional data points may be defined to address potential gaps in the system's performance between retrospective and prospective validation, including without limitation algorithmic discrimination, covariate shift, and prior probability shift.

In one embodiment, the system is updated through being periodically re-trained on new data in process 414. This updated model and its associated metadata are exported via process 306 for use in stent procedures going forward.

In one embodiment, the system extracts predicted expansion metrics alongside angiography clips in process 415 to support training and education for trainees via process 416. Optional comparisons to intravascular imaging-derived labels can be made when available.

In one embodiment, the system includes safeguards for out-of-distribution (OOD) detection, including detecting unseen device types, projection angles, or imaging quality conditions. out-of-distribution detection is implemented by computing a maximum softmax probability, Mahalanobis distance, energy score, or density estimate over the learned video and fused embeddings relative to a training-set reference distribution, by ensemble disagreement, or by a dedicated OOD classifier. If an out-of-distribution condition is detected, the system outputs a warning or suppresses a numeric prediction, instead recommending confirmatory imaging or clinician review.

In one embodiment, the system incorporates image-quality assessment, including detecting severe motion artifacts, poor contrast opacification, or inadequate visualization of the treated segment.

In one embodiment, the system uses DICOM metadata to obtain acquisition parameters such as frame rate, pixel spacing, angulation, magnification, and other parameters useful for standardization.

In one embodiment, the system normalizes pixel spacing and magnification using metadata, calibration objects, or automated calibration approaches, to improve consistency across cases.

In one embodiment, the system optionally incorporates procedural variables available in real time, including without limitation stent size, nominal stent diameter, stent length, balloon diameter, inflation pressure, number of inflations, and use of atherectomy.

In one embodiment, the system predicts a lesion-level or stent-level expansion summary value. In another embodiment, the system predicts a spatially varying expansion estimate across a stent length. In one embodiment, when multiple stents or overlapping stents are present within the field of view, the system predicts a per-stent expansion metric for each identified stent segment or a summary metric for a designated index stent, wherein stent segments are identified by the localization module, by operator input, or by combinations thereof.

In one embodiment, the system predicts an expansion adequacy category corresponding to a probability of under-expansion. In another embodiment, the model outputs an estimate on a normalized scale (0 to 1) that corresponds to a 0% to 100% expansion scale. Other scales may be used.

In one embodiment, the output further includes an uncertainty estimate, confidence level, or risk indicator.

In one embodiment, the system stores the angiography clip, the predicted expansion output, and associated metadata in a database for audit trails and for future model improvement.

In one embodiment, model improvement includes retraining or fine-tuning using additional paired angiography and intravascular imaging cases collected over time, including cases in which clinicians corrected labels or provided additional annotations.

In one embodiment, the system supports multi-center federated learning by aggregating de-identified datasets from multiple institutions, thereby improving generalization.

In one embodiment, privacy-preserving training is performed, including federated learning, differential privacy, or other privacy-preserving techniques, provided that label quality and model robustness requirements are maintained.

In one embodiment, the system is implemented as a software module integrated with existing catheterization laboratory infrastructure, including a workstation or server that interfaces with angiography output and hospital information systems.

In one embodiment, the system is implemented as a standalone appliance with a video capture input and an output display, wherein the appliance provides inference without modification of the angiography console.

In one embodiment, the system outputs are presented as non-binding clinical decision support and do not replace clinician judgment.

In one embodiment, the disclosure is not limited to a particular stent type and can be used with drug-eluting stents, bare-metal stents, bioresorbable scaffolds, and other endovascular implants.

In one embodiment, the disclosure is not limited to coronary arteries and may be applied to other arteries, including without limitation lower extremity arteries, carotid arteries, renal arteries, and intracranial arteries.

In one embodiment, a method for training the model includes: acquiring angiography video data and intravascular imaging data; deriving expansion labels from intravascular imaging; aligning angiography clips with post-stent time windows; encoding clinical variables; training a multi-task model using a weighted loss objective; and storing the trained model.

In one embodiment, a method for inference includes: acquiring a post-stent angiography clip; receiving clinical variables; preprocessing the clip; applying the trained model; generating a predicted expansion metric; and displaying the predicted expansion metric.

In one embodiment, the model is configured such that auxiliary heads are evaluated during training but not necessarily during deployment. In one embodiment, one or more auxiliary outputs are also provided during deployment to assist interpretation.

In one embodiment, the system includes a mapping from model outputs to appropriate thresholds for clinical decision-making, wherein mapping is prescribed by established clinical guidelines.

In one embodiment, the system includes an interface for selecting which angiography run to analyze and for confirming that the analyzed clip corresponds to the post-stent state.

In one embodiment, the system includes an interface for selecting or confirming vessel identity, projection, and stent segment location, wherein user inputs can be used to improve robustness.

In one embodiment, the system is configured to operate under time constraints consistent with PCI workflows, such that predictions are generated quickly enough to be used before the patient leaves the catheterization laboratory.

In one embodiment, the system comprises a memory for storing instructions; one or more processors in communication with the memory, the one or more processors configured to execute the instructions to (a) receive a post-stent cineangiography video clip depicting a stented segment of a blood vessel; (b) receive one or more structured clinical variables associated with a patient corresponding to the post-stent cineangiography video clip; (c) generate, using one or more trained machine learning models, a video embedding based on the post-stent cineangiography video clip; (d) generate, using the one or more trained machine learning models, a clinical embedding based on the one or more structured clinical variables; generate, using the one or more trained machine learning models, a fused embedding based on the video embedding and the clinical embedding; (e) generate, using the one or more trained machine learning models, a predicted stent expansion metric based on the fused embedding, wherein the predicted stent expansion metric corresponds to an intravascular-imaging-derived stent expansion label used to train the one or more trained machine learning models; and (f) provide for output the predicted stent expansion metric for display within a time window that permits operator selection, before the patient leaves a catheterization laboratory, of one or more of: a post-dilation balloon diameter, a post-dilation balloon inflation pressure, or placement of an additional stent.

In some embodiments, the method comprises receiving, by one or more processors, a post-stent cineangiography video clip depicting a stented segment of a blood vessel; receiving, by the one or more processors, one or more structured clinical variables associated with a patient corresponding to the post-stent cineangiography video clip; generating, by the one or more processors using one or more trained machine learning models, a video embedding based on the post-stent cineangiography video clip; generating, by the one or more processors using the one or more trained machine learning models, a clinical embedding based on the one or more structured clinical variables; generating, by the one or more processors using the one or more trained machine learning models, a fused embedding based on the video embedding and the clinical embedding; generating, by the one or more processors using the one or more trained machine learning models, a predicted stent expansion metric based on the fused embedding, wherein the predicted stent expansion metric corresponds to an intravascular-imaging-derived stent expansion label used to train the one or more trained machine learning models; and providing for output, by the one or more processors, the predicted stent expansion metric for display to a user within a time window that permits operator selection, before the patient leaves a catheterization laboratory, of one or more of: a post-dilation balloon diameter, a post-dilation balloon inflation pressure, and/or placement of an additional stent.

In other embodiments, the method comprises obtaining a training dataset comprising (i) a plurality of post-stent cineangiography video clips and (ii) corresponding intravascular-imaging-derived stent expansion labels; training, by one or more processors, a machine learning model using the training dataset to predict a stent expansion metric from a cineangiography video clip and one or more structured clinical variables, wherein the machine learning model is trained using a multi-task objective that includes a primary loss associated with the stent expansion metric and one or more auxiliary losses associated with one or more auxiliary prediction tasks, and wherein the trained machine learning model is configured to generate the stent expansion metric on a timescale consistent with operator selection of a stent optimization action before a patient leaves a catheterization laboratory; and storing, by the one or more processors, the trained machine learning model in electronic memory for later deployment to predict stent expansion without requiring intravascular imaging input at inference time.

Some portions of the detailed description are presented in terms of algorithms and symbolic representations of operations on data within a computer memory. These algorithmic descriptions and representations can be used by those skilled in the computer and software related fields.

Unless specifically stated otherwise, it is appreciated that throughout the description, discussions utilizing terms such as “processing,” “computing,” “training,” “encoding,” “predicting,” “classifying,” “segmenting,” “determining,” “generating,” “displaying,” and the like, refer to the action and processes of a computer system, AI processor, GPU, or electronic device, that manipulates and transforms data represented as physical (electronic) quantities within the computer system's registers and memories into other data similarly represented as physical quantities.

The present disclosure, in some embodiments, also relates to apparatus for performing the operations herein. This apparatus may be specially constructed for the required purposes, or it may comprise a general-purpose computer selectively activated or reconfigured by a computer program stored in the computer.

The required structure for a variety of these systems will appear from the description herein. The present disclosure is not described with reference to any particular programming language, and various embodiments may thus be implemented using a variety of programming languages and machine learning frameworks.

The aspects, embodiments, features, and examples of the disclosure are to be considered illustrative in all respects and are not intended to limit the disclosure, the scope of which is defined only by the claims. Other embodiments, modifications, and usages will be apparent to those skilled in the art without departing from the spirit and scope of the claimed disclosure.

The use of headings and sections in the application is not meant to limit the disclosure; each section can apply to any aspect, embodiment, or feature of the disclosure. Only those claims which use the words “means for” are intended to be interpreted under 35 USC 112(f). Absent a recital of “means for” in the claims, such claims should not be construed under 35 USC 112(f). Limitations from the specification are not intended to be read into any claims, unless such limitations are expressly included in the claims.

When values or ranges of values are given, each value and the end points of a given range and the values therebetween may be increased or decreased by 20%, while still staying within the teachings of the disclosure, unless some different range is specifically mentioned.

Throughout the application, where compositions are described as having, including, or comprising specific components, or where processes are described as having, including or comprising specific process steps, it is contemplated that compositions of the present teachings also consist essentially of, or consist of, the recited components, and that the processes of the present teachings also consist essentially of, or consist of, the recited process steps.

It will be appreciated that several of the above-disclosed and other features and functions, or alternatives or varieties thereof, may be desirably combined into many other different processes or applications. Also, that various alternatives, modifications, variations or improvements therein may be subsequently made by those skilled in the art which are also intended to be encompassed by the following claims.

In the description above, for the purposes of explanation, numerous specific requirements and several specific details have been set forth to provide a thorough understanding of the embodiments. It will be apparent however, to one skilled in the art, that one or more other embodiments may be practiced without some of these specific details. The embodiments described are not provided to limit the invention, but to illustrate it. The scope of the invention is not to be determined by the specific examples provided above. In other instances, well-known structures and processes have been shown in block diagram form or without detail in order to avoid obscuring the understanding of the description.

It should also be appreciated that reference throughout this specification to “one embodiment”, “an embodiment”, “one or more embodiments”, or “different embodiments”, for example, means that a particular feature may be included in the practice of the invention. Similarly, it should be appreciated that in the description various features are sometimes grouped together in a single embodiment, figure, or description thereof for the purpose of streamlining the disclosure and aiding in the understanding of various inventive aspects. This method of disclosure, however, is not to be interpreted as reflecting an intention that the invention requires more features than are expressly recited in each claim. Rather, as the following claims reflect, inventive aspects may lie in less than all features of a single disclosed embodiment. In another situation, an inventive aspect may include a combination of embodiments described herein or in a combination of less than all aspects described in a combination of embodiments.

1. A method, comprising:
receiving, by one or more processors, a post-stent cineangiography video clip depicting a stented segment of a blood vessel;
receiving, by the one or more processors, one or more structured clinical variables associated with a patient corresponding to the post-stent cineangiography video clip;
generating, by the one or more processors using one or more trained machine learning models, a video embedding based on the post-stent cineangiography video clip;
generating, by the one or more processors using the one or more trained machine learning models, a clinical embedding based on the one or more structured clinical variables;
generating, by the one or more processors using the one or more trained machine learning models, a fused embedding based on the video embedding and the clinical embedding;
generating, by the one or more processors using the one or more trained machine learning models, a predicted stent expansion metric based on the fused embedding, wherein the predicted stent expansion metric corresponds to an intravascular-imaging-derived stent expansion label used to train the one or more trained machine learning models; and
providing for output, by the one or more processors, the predicted stent expansion metric for display to a user within a time window that permits operator selection, before the patient leaves a catheterization laboratory, of one or more of: a post-dilation balloon diameter, a post-dilation balloon inflation pressure, and/or placement of an additional stent.

2. The method of claim 1, wherein the intravascular-imaging-derived stent expansion label is derived from intravascular optical coherence tomography (OCT), intravascular ultrasound (IVUS), or another intravascular imaging modality.

3. The method of claim 1, wherein the method is performed without acquiring intravascular imaging data at a time of generating the predicted stent expansion metric.

4. The method of claim 1, further comprising preprocessing the post-stent cineangiography video clip by one or more of: selecting a fixed-length frame sequence from a longer angiography run; resizing frames to a predetermined resolution; normalizing pixel intensities; cropping frames to a region of interest corresponding to the stented segment;
or sampling frames to a predetermined number of frames.

5. The method of claim 1, wherein the post-stent cineangiography video clip comprises between about 4 seconds and about 10 seconds of angiography video.

6. The method of claim 5, wherein a number of frames sampled from the post-stent cineangiography video clip is determined as a function of an acquisition frame rate of the post-stent cineangiography video clip.

7. The method of claim 1, wherein the one or more structured clinical variables include one or more of: age, sex, diabetes status, dyslipidemia, chronic kidney disease status, dialysis history, smoking status, or prior coronary artery bypass grafting.

8. The method of claim 1, wherein generating the predicted stent expansion metric comprises outputting a continuous value representing a percent stent expansion.

9. The method of claim 1, wherein generating the predicted stent expansion metric comprises outputting an ordered category of stent expansion adequacy.

10. The method of claim 9, wherein the ordered category is selected from a set comprising at least subpar, adequate, and optimal.

11. The method of claim 1, wherein receiving the post-stent cineangiography video clip comprises receiving a single-view cineangiography video clip acquired in a standard angiographic projection.

12. The method of claim 1, wherein receiving the post-stent cineangiography video clip comprises receiving a plurality of cineangiography video clips acquired in different projections, and wherein generating the predicted stent expansion metric comprises fusing features from the plurality of cineangiography video clips.

13. The method of claim 12, wherein fusing features from the plurality of cineangiography video clips comprises providing C-arm angulation metadata corresponding to each cineangiography video clip as an input to a fusion mechanism.

14. The method of claim 1, further comprising generating, by the one or more processors, an alert when the predicted stent expansion metric is below a predetermined threshold.

15. The method of claim 14, further comprising receiving, in response to the alert, an additional post-stent cineangiography video clip acquired after a stent optimization intervention; and generating, using the one or more trained machine learning models, an updated predicted stent expansion metric based on the additional post-stent cineangiography video clip.

16. The method of claim 1, further comprising storing, in electronic memory, one or more of: (i) the post-stent cineangiography video clip, (ii) the predicted stent expansion metric, and (iii) metadata associated with the post-stent cineangiography video clip.

17. A method, comprising:
receiving, by one or more processors, a post-stent cineangiography video clip depicting a stented segment of a blood vessel;
receiving, by the one or more processors, one or more structured clinical variables associated with a patient corresponding to the post-stent cineangiography video clip;
generating, by the one or more processors using one or more trained machine learning models, a video embedding based on the post-stent cineangiography video clip;
generating, by the one or more processors using the one or more trained machine learning models, a clinical embedding based on the one or more structured clinical variables;
generating, by the one or more processors using the one or more trained machine learning models, a fused embedding based on the video embedding and the clinical embedding;
generating, by the one or more processors using the one or more trained machine learning models, a predicted stent expansion metric based on the fused embedding, wherein the predicted stent expansion metric corresponds to an intravascular-imaging-derived stent expansion label used to train the one or more trained machine learning models;
providing for output, by the one or more processors, the predicted stent expansion metric for display to a user within a time window that permits operator selection, before the patient leaves a catheterization laboratory, of one or more of: a post-dilation balloon diameter, a post-dilation balloon inflation pressure, and/or placement of an additional stent; and
preprocessing the post-stent cineangiography video clip by one or more of: selecting a fixed-length frame sequence from a longer angiography run; resizing frames to a predetermined resolution; normalizing pixel intensities; cropping frames to a region of interest corresponding to the stented segment; or sampling frames to a predetermined number of frames;
wherein the intravascular-imaging-derived stent expansion label is derived from intravascular optical coherence tomography (OCT), intravascular ultrasound (IVUS), or another intravascular imaging modality.

18. The method of claim 17, wherein generating the predicted stent expansion metric comprises outputting an ordered category of stent expansion adequacy.

19. A method, comprising:
receiving, by one or more processors, a post-stent cineangiography video clip depicting a stented segment of a blood vessel;
receiving, by the one or more processors, one or more structured clinical variables associated with a patient corresponding to the post-stent cineangiography video clip;
generating, by the one or more processors using one or more trained machine learning models, a video embedding based on the post-stent cineangiography video clip;
generating, by the one or more processors using the one or more trained machine learning models, a clinical embedding based on the one or more structured clinical variables;
generating, by the one or more processors using the one or more trained machine learning models, a fused embedding based on the video embedding and the clinical embedding;
generating, by the one or more processors using the one or more trained machine learning models, a predicted stent expansion metric based on the fused embedding, wherein the predicted stent expansion metric corresponds to an intravascular-imaging-derived stent expansion label used to train the one or more trained machine learning models;
providing for output, by the one or more processors, the predicted stent expansion metric for display to a user within a time window that permits operator selection, before the patient leaves a catheterization laboratory, of one or more of: a post-dilation balloon diameter, a post-dilation balloon inflation pressure, and/or placement of an additional stent; and
preprocessing the post-stent cineangiography video clip by one or more of: selecting a fixed-length frame sequence from a longer angiography run; resizing frames to a predetermined resolution; normalizing pixel intensities; cropping frames to a region of interest corresponding to the stented segment; or sampling frames to a predetermined number of frames;
wherein the method is performed without acquiring intravascular imaging data at a time of generating the predicted stent expansion metric.

20. The method of claim 19, wherein receiving the post-stent cineangiography video clip comprises receiving a plurality of cineangiography video clips acquired in different projections, and wherein generating the predicted stent expansion metric comprises fusing features from the plurality of cineangiography video clips.
